(19) United States
(12) Patent Application Publication
(10) Pub. No.: US 2025/0314670 A1
(43) Pub. Date: Oct. 9, 2025

(54) TORSIONAL GUIDED WAVE-BASED BLOOD VISCOELASTICITY MEASUREMENT DEVICE AND METHOD USING CAPILLARY METAL TUBE

(71) Applicants: Zhejiang University, Hangzhou (CN); Huzhou Institute of Zhejiang University, Huzhou (CN)

(72) Inventors: Zhifeng TANG, Hangzhou (CN); Fengjiang ZHANG, Hangzhou (CN); Eryu YANG, Hangzhou (CN); Jianjun WU, Huzhou (CN); Min YAN, Hangzhou (CN); Fuzai LV, Hangzhou (CN)

(73) Assignees: Zhejiang University, Hangzhou (CN); Huzhou Institute of Zhejiang University, Huzhou (CN)

(21) Appl. No.: 19/206,116

(22) Filed: May 13, 2025

Related U.S. Application Data
(63) Continuation of application No. PCT/CN2023/118325, filed on Sep. 12, 2023.

(30) Foreign Application Priority Data
Nov. 14, 2022 (CN) 202211425810.2

(51) Int. Cl. G01N 33/86; B01L 3/00

(52) U.S. Cl. CPC G01N 33/86; B01L 3/502715; B01L 2300/027; B01L 2300/0838; B01L 2300/18; B01L 2400/043

(57) ABSTRACT

The provided is a torsional guided wave-based blood viscoelasticity measurement device and method using a capillary metal tube. The device includes a housing, a capillary metal tube, a signal receiving module, and a signal excitation module, where the capillary metal tube is disposed inside the housing and configured to hold a blood sample; the signal excitation module is disposed at one end of the capillary metal tube and includes a cuboid permanent magnet and an electrode; the signal receiving module is disposed at the other end of the capillary metal tube and includes a cylindrical permanent magnet and a receiving coil; an excitation is performed on the capillary metal tube to generate a torsional guided wave; when a fluid viscosity in the tube changes, energy attenuation of the guided wave propagating along the tube differs; and the guided wave energy attenuation is defined to reflect a relative blood viscosity.

### CROSS-REFERENCE TO THE RELATED APPLICATIONS

This application is a continuation application of International Application No. PCT/CN2023/118325, filed on Sep. 12, 2023, which is based upon and claims priority to Chinese Patent Application No. 202211425810.2, filed on Nov. 14, 2022, the entire contents of which are incorporated herein by reference.

### TECHNICAL FIELD

The present disclosure belongs to the field of medical instruments, and relates to a blood measurement device and method, in particular to a torsional ultrasonic guided wave-based blood viscoelasticity measurement device and method.

### BACKGROUND

Blood viscoelasticity is an important property of human blood. Its measurement generally involves monitoring, measuring, and analyzing viscoelastic changes during coagulation to fibrinolysis using a thromboelastograph (TEG), and qualitatively and quantitatively predicting blood sample coagulation to obtain a thrombelastogram curve. Therefore, viscoelasticity can be used to assist physicians in accurately assessing patients' coagulation function. Currently, the main equipment for blood viscoelasticity measurement is TEG. The basic measurement principle for TEG provided by Chinese Patent Application CN208188122 primarily involves detecting the motion state of a probe in a blood sample within a measurement cup via an electromagnetic sensor to evaluate blood viscoelasticity. This TEG has the following limitations in addition to high costs. The required blood sample volume is large, with each measurement taking over 50 minutes, leading to generally prolonged measurement durations. Exposure of the blood sample to air during measurement may compromise the accuracy of measurement results. Additionally, the rotational immersion of the probe into the blood sample can interfere with the coagulation process of the blood sample.

In summary, traditional blood viscoelasticity measurement technologies struggle to meet demands of broader application scenarios such as infant/child patients, perioperative bedside rapid testing, home-based routine monitoring, and emergency field test. Therefore, there is an urgent need to develop a micro-volume, rapid, and non-immersive blood viscoelasticity measurement technology.

Blood viscoelasticity changes can be measured based on an ultrasonic guided wave in a sealed capillary metal tube. This method achieves higher accuracy and shortens the coagulation time of the blood sample due to the reduced blood sample volume required. According to the Study on Propagation Characteristics of Torsional Ultrasonic Guided Waves in Tubes with Viscoelastic Coatings, torsional guided waves exhibit higher sensitivity to fluid viscosity changes in tubes compared to longitudinal guided waves. However, the excitation of torsional guided waves imposes significant requirements on the device. Specifically, traditional excitation methods involve wrapping an iron-cobalt alloy strip and a coil around the tube to form a transducer, but the inherent flexibility of the iron-cobalt strip makes it hard to assemble on the capillary metal tube with an extremely small outer diameter. In Chinese Patent Application CN113203661A, a magnetostrictive powder coating is applied to a capillary metal tube, but this approach has the following issues. The required process is complex and costly. The non-uniform bias magnetic field distribution introduces susceptibility to excitation signal interference during signal reception, resulting in less pure torsional guided waves and unstable waveforms. The machine learning (ML) method demands extensive datasets and prolonged training periods, while the absence of temperature measurement compromises the accuracy of blood viscosity values. Furthermore, this approach only captures static blood viscosity values, failing to enable dynamic blood viscoelasticity measurement or analyze time-dependent blood viscosity changes.

In conclusion, currently, there is no technical solution that can excite pure torsional guided waves in capillary metal tubes.

### SUMMARY

To address the problems of blood viscoelasticity measurement described in the background section, an objective of the present disclosure is to provide a torsional guided wave-based blood viscoelasticity measurement device and method using a capillary metal tube. The present disclosure utilizes an ultrasonic guided wave to measure a blood sample in a sealed capillary metal tube, enabling excitation of a pure torsional guided wave to achieve micro-volume, rapid, and non-immersive blood viscoelasticity measurement.

The present disclosure adopts the following technical solutions.

I. A torsional guided wave-based blood viscoelasticity measurement device using a capillary metal tube includes:
a housing, a capillary metal tube, a signal receiving module, and a signal excitation module, where
the capillary metal tube is disposed inside the housing, and is configured to hold a blood sample;
the signal excitation module is disposed at one end of the capillary metal tube, and includes a cuboid permanent magnet and an electrode; the cuboid permanent magnet is configured to provide a static bias magnetic field; and the electrode is configured to conduct a current and apply a dynamic induced magnetic field to the capillary metal tube; and
the signal receiving module is disposed at the other end of the capillary metal tube, and includes a cylindrical permanent magnet and a receiving coil; the cylindrical permanent magnet is disposed at an end portion of the capillary metal tube; and the receiving coil is disposed close to the end portion of the capillary metal tube.

The device further includes a microcontroller, a guided wave excitation device, a pulse generation device, a power amplification device, an echo receiving module, a preamplifier module, a data acquisition module, and a display module, where the receiving coil is connected to the microcontroller through the echo receiving module, the preamplifier module, and the data acquisition module in sequence; the microcontroller is connected to the electrode through the guided wave excitation device, the pulse generation device, and the power amplification device in sequence; and the microcontroller is connected to the display module.

The housing is further internally provided with a heating layer, an insulation layer, a temperature probe, and a temperature controller;
the heating layer is disposed outside the capillary metal tube, is connected to the capillary metal tube through contact, and is electrically connected to the microcontroller;
the insulation layer contacts and wraps around the heating layer;
the temperature probe is disposed at the one end of the capillary metal tube where the electrode is located; and
the temperature controller is electrically connected to the microcontroller through the temperature probe.

The signal receiving module is disposed at the other end of the capillary metal tube; the cylindrical permanent magnet is directly connected to an end face of the capillary metal tube through magnetism; the receiving coil is sleeved outside the capillary metal tube, such that an inner wall of the coil does not contact an outer wall of the metal tube, and a difference between an inner diameter of the coil and an outer diameter of the metal tube is less than 1.0 mm; and the capillary metal tube is directly detachable and replaceable through a plug-and-pull method.

The capillary metal tube is made of a magnetic material; and the cylindrical permanent magnet is magnetically adsorbed onto the end portion of the capillary metal tube.

The capillary metal tube is made of a material, including but not limited to pure nickel, carbon steel, iron-cobalt alloy, iron-aluminum alloy, and iron-cobalt-nickel alloy.

The end portion of the capillary metal tube is provided with a rubber plug for sealing.

The present disclosure provides the cuboid permanent magnet and the cylindrical permanent magnet to provide the static bias magnetic field for the excitation and receiving units, differing from the annular magnetic field formed by permanent magnets arranged at two sides in the prior art. The axial static bias magnetic field provided by the cuboid permanent magnet exhibits higher uniformity, generating a purer torsional guided wave. Compared with transmitter-receiver-integrated magnetostrictive excitation technologies, the transmitter-receiver-separated signal acquisition method reduces excitation interference coupled to the received signal, resulting in a more stable waveform and a higher signal-to-noise ratio (SNR).

II. A torsional guided wave-based blood viscoelasticity measurement method using a capillary metal tube includes the following steps:
1) calculating a guided wave dispersion curve of the capillary metal tube based on a structural geometric parameter and a material mechanical characteristic of the capillary metal tube; and selecting an excitation frequency based on the guided wave dispersion curve;

In a specific implementation, to ensure sufficient acoustic field intensity and prevent packet overlap, the single excitation pulse count does not exceed 4. The excitation frequency is determined by combining wavelength (defined by the length of the capillary metal tube) and wave velocity.
2) exciting the electrode on the capillary metal tube when the capillary metal tube is empty, and generating a torsional guided wave that propagates reciprocally between two end faces of the capillary metal tube until energy is depleted, where the tube has a length of l m; collecting, by the receiving coil, a total of 2 klm guided wave signals from first k reciprocations as an empty-tube reference signal w₀;
3) filling a blood sample into the capillary metal tube; acquiring an echo signal in the same manner as the step 2) as a blood-filled test signal, where an i-th blood-filled test signal is denoted as wᵢ,
4) extracting envelopes from the empty-tube reference signal w₀ and each blood-filled test signal wᵢ respectively to obtain envelope signals; taking n echo peaks from each of the envelope signals for fitting, and obtaining attenuation rates b₀ and b₁ of the echo peaks, respectively; and determining, based on the attenuation rates b₀ and b₁ and a pre-calibrated relationship between a relative blood viscosity of the blood sample and the attenuation rate, a relative blood viscosity corresponding to each blood-filled test signal wᵢ; and

In the present disclosure, a positive correlation is established between the relative blood viscosity of the blood sample and the attenuation rate.
5) setting a sampling count m and a sampling interval Δt; filling the blood sample into the capillary metal tube, and continuously repeating the steps 3) and 4) to perform sampling and obtain a relative blood viscosity for each test; and plotting a time-dependent relative blood viscosity curve of the blood sample over a duration of m*Δt as a thrombelastogram, thereby achieving a blood viscoelasticity measurement.

The innovation of the present disclosure lies in deriving the blood viscoelasticity measurement result through the blood sample measurement using the capillary metal tube. The present disclosure obtains accurate attenuation rates through specific echo peak processing of the measured echo signal. The present disclosure discovers and establishes the relationship between the attenuation rate and the relative blood viscosity of the blood sample, enabling a precise blood viscoelasticity measurement result based on this relationship.

In the step 4), the taking n echo peaks from each of the envelope signals specifically includes: selecting, based on the envelope signal, suitable first threshold l₁ and second threshold l₂, with an interval being l=l₁−l₂; establishing, starting from an initial point x₀ of the envelope signal, n consecutive segments, each with a length of l, where the segments are [x₀,x₀+l], [x₀+l, x₀+2l], . . . , [x₀+ (n−1)l, x₀+nl], and each segment includes only one peak; and extracting the peak from each segment as an echo peak, and extracting all peaks from the n segments as finally selected n echo peaks.

In the step 4), the attenuation rate is derived by fitting as follows:

$$y = a * {b}^{x}$$

where, x denotes a sequence number of the echo peak; y denotes a value of the echo peak; b denotes the attenuation rate of the echo peak; and a denotes an amplitude normalization coefficient.

The method of the present disclosure acquires the echo peak attenuation rate through specific processing, which more accurately reflects the blood viscoelasticity parameter compared with amplitude attenuation and time-of-flight (ToF) measurements in conventional techniques. The present disclosure exclusively establishes a nonlinear relationship between the echo peak attenuation rate and the relative blood viscosity, enabling a precise blood viscoelasticity measurement result.

The fundamental principle of the present disclosure is as follows. When a target blood sample is injected into the capillary metal tube, an alternating current applied to the electrode sleeved outside the capillary tube generates a dynamic magnetic field. The dynamic magnetic field interacts with the bias magnetic field from the cuboid permanent magnet, exciting a torsional ultrasonic guided wave through the magnetostrictive effect. Partial wave energy leaks into the blood sample during propagation along the capillary tube due to blood viscosity, thereby causing attenuation of the ultrasonic guided wave during propagation. As the blood gradually coagulates and undergoes fibrinolysis in a static condition, the blood viscosity changes accordingly. The time-dependent change of the guided wave attenuation rate in the capillary tube is plotted, and it is combined with the fundamental relationship between the blood viscosity and the guided wave attenuation rate to plot the thrombelastogramof the blood sample.

The present disclosure has the following beneficial effects:

The present disclosure excites a torsional guided wave in a blood-filled capillary metal tube via a magnetostrictive effect, featuring short overall measurement time, micro-volume blood sample, and air-free contact that avoids interference with coagulation of the blood sample. The present disclosure has the advantages of simple device, easy operation, low cost, and high precision, and solves the problem of prolonged measurement time in blood viscoelasticity measurement. The present disclosure is suitable for broader applications including infant/child patients, perioperative bedside rapid testing, home-based routine monitoring, and emergency field test.

### BRIEF DESCRIPTION OF THE DRAWINGS

FIG. 1 is a structural diagram of a blood viscoelasticity measurement device;

FIG. 2 is a schematic diagram of a signal transceiver module;

FIG. 3 is a structural schematic diagram of a temperature control module;

FIG. 4 is a partial structural schematic diagram of a housing;

FIG. 5 shows an original signal from a metal tube filled with a blood sample;

FIG. 6 is a schematic diagram of an envelope of a blood-filled test signal and a set threshold; and

FIG. 7 shows a relationship between an attenuation rate of the blood sample and a time.

Reference Numerals: 1. cylindrical permanent magnet; 2. receiving coil; 3. electrode; 4. cuboid permanent magnet; 5. capillary metal tube; 6. temperature probe; 7. cover plate; 8. heating layer; 9. insulation layer; 10. first socket; 11. second socket; 12. front end cover; 13. housing; 14. base platform; 15. rear end cover; 16. microcontroller; 17. guided wave excitation device; 18. pulse generation device; 19. power amplification device; 20. echo receiving module; 21. preamplifier module; 22. data acquisition module; 23. display module; 24. temperature controller; and 25. rubber plug.

### DETAILED DESCRIPTION OF THE EMBODIMENTS

The present disclosure will be further described in detail below in conjunction with the drawings and embodiments.

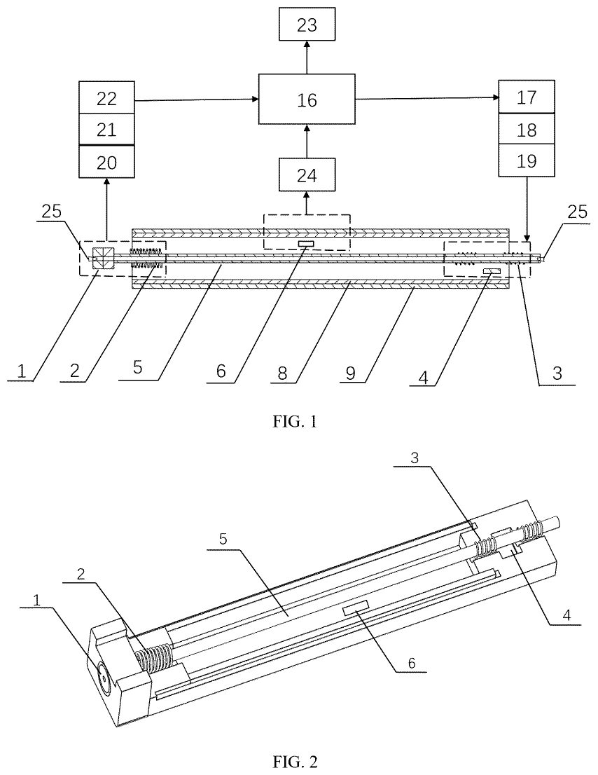

As shown in FIG. 1, a device of the present disclosure includes housing 13, capillary metal tube 5, a signal receiving module, and a signal excitation module.

The capillary metal tube 5 is disposed inside the housing 13, serves as a test fluid container and guided wave propagation carrier, and holds an in-vitro blood sample.

The signal excitation module is disposed at one end of the capillary metal tube 5, and includes cuboid permanent magnet 4 and electrode 3. The cuboid permanent magnet 4 is configured to provide a static bias magnetic field. The electrode 3 is configured to conduct a current and apply a dynamic induced magnetic field to the capillary metal tube 5. The cuboid permanent magnet 4 may not contact the capillary metal tube 5. The electrode 3 is provided to apply a circumferential dynamic magnetic field through the current, and it reduces contact with the capillary metal tube, thereby avoiding interference to guided wave propagation in the tube.

The signal receiving module is disposed at the other end of the capillary metal tube 5, and includes cylindrical permanent magnet 1 and receiving coil 2. The cylindrical permanent magnet 1 is configured to provide a torsional constant magnetic field. The receiving coil 2 is configured to receive an echo signal when energized. The cylindrical permanent magnet 1 is disposed at an end portion of the capillary metal tube 5 to provide the torsional constant magnetic field. The receiving coil 2 is disposed close to the end portion of the capillary metal tube 5 and may be sleeved outside the capillary metal tube 5 without contact.

There is a gap between an inner diameter of the receiving coil 2 and an outer diameter of the capillary metal tube 5, and the gap does not exceed 2 mm.

The device further includes microcontroller 16, guided wave excitation device 17, pulse generation device 18, power amplification device 19, echo receiving module 20, preamplifier module 21, data acquisition module 22, and display module 23. The receiving coil 2 is connected to the microcontroller 16 through the echo receiving module 20, the preamplifier module 21, and the data acquisition module 22 in sequence. The microcontroller 16 is connected to the electrode 3 through the guided wave excitation device 17, the pulse generation device 18, and the power amplification device 19 in sequence. The microcontroller 16 is connected to the display module 23.

The microcontroller 16 generates an initial excitation pulse signal. The initial excitation pulse signal is converted into an analog signal through the guided wave excitation device 17 and the pulse generation device 18 in sequence. The analog signal is power-amplified by the power amplification device 19 and then input to the electrode 3, thereby generating an alternating current for producing the dynamic magnetic field. The dynamic magnetic field combines with the bias magnetic field generated by the cuboid permanent magnet 4 to form a torsional guided wave. The torsional guided wave is coupled to the capillary metal tube 5 and propagates reciprocally along the capillary metal tube 5.

An echo signal of the torsional guided wave is received by the signal receiving module including the receiving coil 2 and the cylindrical permanent magnet 1. The echo signal is sequentially received by the echo receiving module 20, amplified by the preamplifier module 21, and acquired by the data acquisition module 22 to achieve sampling reception. The echo signal is then transmitted to the microcontroller 16 for data processing. A single-measurement attenuation rate is derived by performing power function fitting on an echo peak of an original signal. Through the above test method, a time-dependent change of a blood viscosity converted from the guided wave attenuation rates is displayed on the display module 23.

The initial excitation pulse signal has a dominant frequency not exceeding 500 kHz and includes no more than 4 cycles.

The housing 13 is further internally provided with heating layer 8, insulation layer 9, temperature probe 6, and temperature controller 24.

The heating layer 8 surrounds the capillary metal tube 5. The heating layer is connected to the capillary metal tube 5 through contact and is electrically connected to the microcontroller 16.

The insulation layer 9 contacts and wraps around the heating layer 8. The insulation layer 9 covers a surface of the heating layer 8 to provide thermal insulation and buffering effects.

The temperature probe 6 is disposed at the one end of the capillary metal tube 5 where the electrode 3 is located.

The temperature controller 24 is electrically connected to the microcontroller 16 through the temperature probe 6.

A temperature of the capillary metal tube 5 is collected in real time through the temperature probe 6, and is transmitted to the microcontroller 16 via the temperature controller 24 so as to control the operation of the heating layer 8. Heating module 7 is driven to maintain a constant 37° C. temperature like that of human blood in case of a temperature change. By maintaining the constant temperature, the design simulates blood conditions in the human body.

The signal receiving module is disposed at the other end of the capillary metal tube 5. The cylindrical permanent magnet 1 is directly connected to an end face of the capillary metal tube 5 through magnetism. The receiving coil 2 is sleeved outside the capillary metal tube 5. An inner wall of the coil does not contact an outer wall of the metal tube, and a difference between the inner diameter of the coil and the outer diameter of the metal tube is less than 1.0 mm. The capillary metal tube 5 is directly detachable and replaceable through a plug-and-pull method.

The capillary metal tube 5 is made of a magnetic material. The cylindrical permanent magnet 1 is magnetically adsorbed onto the end portion of the capillary metal tube 5. The end face of the capillary metal tube 5 undergoes precision machining to ensure perpendicularity to an axis of the metal tube and absence of burrs or notches, guaranteeing repeatability of static magnetic field loading and optimal guided wave reflection.

The capillary metal tube 5 is made of a magnetostrictive material or a magnetic conductive metal material, including but not limited to pure nickel, carbon steel, iron-cobalt alloy, iron-aluminum alloy, and iron-cobalt-nickel alloy.

The capillary metal tube 5 has a capillary inner diameter and a length not exceeding 200 mm, with the inner diameter not exceeding 1.5 mm.

The cylindrical permanent magnet 1 is axially magnetized, while the cuboid permanent magnet 4 is magnetized along a thickness direction thereof, both with grades of N35 or higher. The cylindrical permanent magnet 1 is perforated and coaxially aligned with the capillary metal tube 5 during mounting. The design enables observation of a blood sample outflow during injection to verify complete filling of the capillary metal tube 5.

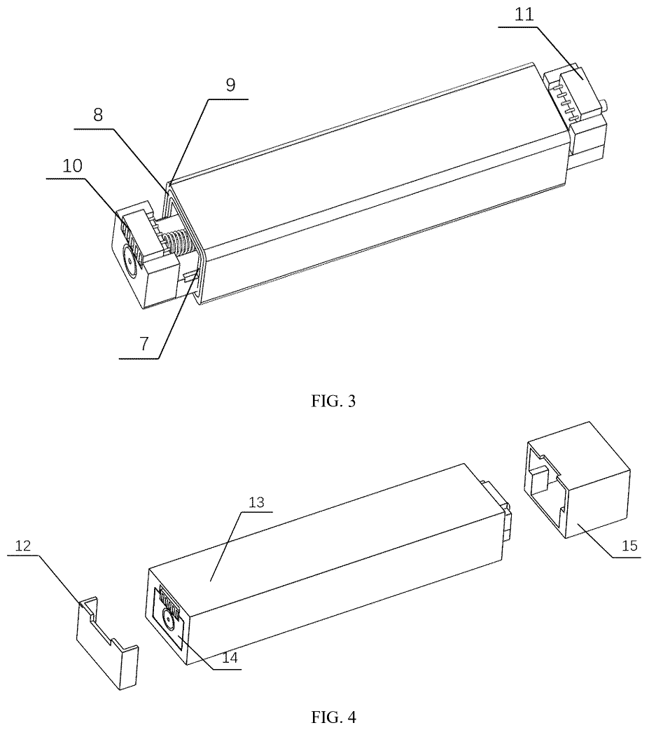

In a specific implementation, as shown in FIGS. 2 to 4, the housing 13 internally includes base platform 14 and cover plate 7, and externally includes front end cover 12 and rear end cover 15. The base platform 14 incorporates the heating layer 8 and the insulation layer 9. Two ends of the base platform 14 are provided with first socket 10 and second socket 11 for external connections. The first socket 10 and the second socket 11 are electrically connected to the receiving coil 2 and the electrode 3 respectively. The base platform 14 is provided in an inner cavity of the housing 13 after being covered with the upper cover plate 7. The housing 13 includes two ends provided with the front end cover 12 and the rear end cover 15, respectively.

The signal excitation module and the receiving module both are disposed on the base platform. The cuboid permanent magnet is placed parallel to the tube in a groove of the base platform. The electrode 3 is connected to the second socket 11 through a wire, sleeved at one end of the capillary metal tube 5, and located at two sides of the cuboid permanent magnet 4. The receiving coil 2 in the signal receiving module is connected to the first socket 10 through a wire. The cylindrical permanent magnet 1 is adsorbed onto the end face of the capillary metal tube 5.

In the temperature control module, the temperature probe 6 is attached to an inner wall of the base platform and connected to the first socket 10 through a wire. The mounting process should avoid affecting the disassembly/assembly and operation of the capillary metal tube 5. The heating layer 8 covers a surface of the cover plate 7. The insulation layer 9 covers the surface of the heating layer 8 to provide thermal insulation and buffering effects.

The housing 13 is sleeved outside the insulation layer, with an end face flush with one end of the magnet to achieve fixation. The front end cover 12 and the rear end cover 14 adhered to insulation rubber are located on front and rear end faces of the housing 13.

An end portion of the capillary metal tube 5 is provided with rubber plug 25 for sealing. The rubber plug 25 matches an inner hole of the capillary metal tube 5 in terms of dimensions. The rubber plug 25 ensures that the blood sample does not contact air after filling.

A small amount of patient blood extracted is injected from an inlet end (signal excitation end) of the capillary metal tube 5 until there is a blood sample overflow at an outlet end (cylindrical permanent magnet 1). A surface of the cylindrical permanent magnet 1 is sealed with rubber plug 25. After a syringe is removed, the rubber plug 25 blocks the inlet end. The measurement can commence upon completion of sealing.

A testing procedure of the present disclosure is as follows:

For a capillary carbon steel tube with an inner diameter of 1.5 mm, an outer diameter of 3 mm, a length of 200 mm, a density of 7,800 kg/m³, an elastic modulus of 210 GPa, and a Poisson's ratio of 0.28:
1) A guided wave dispersion curve of the capillary metal tube 5 is calculated based on the structural geometric parameters and material mechanical characteristics of the capillary metal tube 5.

The length of the capillary metal tube selected is 200 mm and the single excitation pulse cycles count 4 to ensure sufficient acoustic field intensity. To prevent wave packet overlap, the excitation pulse wavelength does not exceed 25 mm. Therefore, the minimum excitation frequency is calculated as 128 kHz from the zeroth-order torsional wave velocity (3,250 m/s).
2) When the capillary metal tube 5 is empty, the receiving coil 2 on the capillary metal tube 5 is excited to generate a torsional guided wave propagating along the capillary metal tube 5. An echo signal propagating reciprocally over 10 m on the capillary metal tube 5 is collected as an empty-tube reference signal.
3) A blood sample is injected into the excitation end of the capillary metal tube 5 using a syringe connected to a flexible tube. When there is a blood sample outflow at the receiving-end magnet, the tube is considered fully filled. Both ends are sealed with rubber plugs 25. An echo signal is acquired in the same manner as the step 2) as a blood-filled test signal.

The method of the present disclosure features rapid transport speed. In the step 4 of the method, relative blood viscosity values collected at 5 s intervals are updated in real time on the display module 10 without requiring completion of all sampling before displaying results.

The device of the present disclosure remains horizontal during measurement to prevent sedimentation of red blood cells in the blood sample from affecting final results.

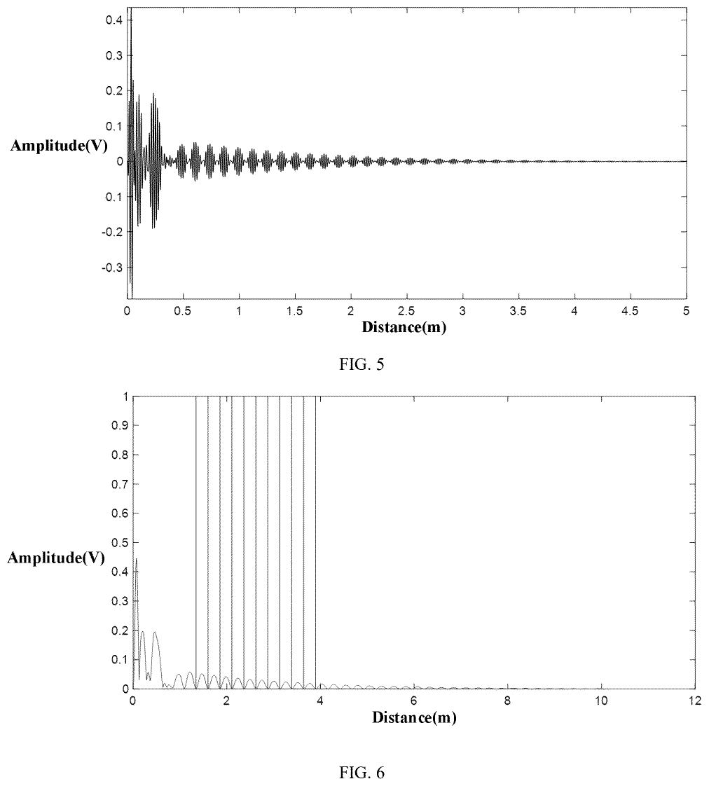

As shown in FIG. 5, the original echo signal of single sampling exhibits amplitude attenuation with increasing propagation distance. The single-measurement attenuation rate is derived by performing power function fitting on the echo peak of the original signal. Through the aforementioned test method, the time-dependent change of the relative blood viscosity converted from the guided wave attenuation rate is displayed on the display module 23.
4) A signal envelope is plotted with a threshold setting, and echo peaks from 10 segments are selected as shown in FIG. 6. The signal attenuation rate and relative blood viscosity are calculated through 360 consecutive samplings with 5 s intervals.

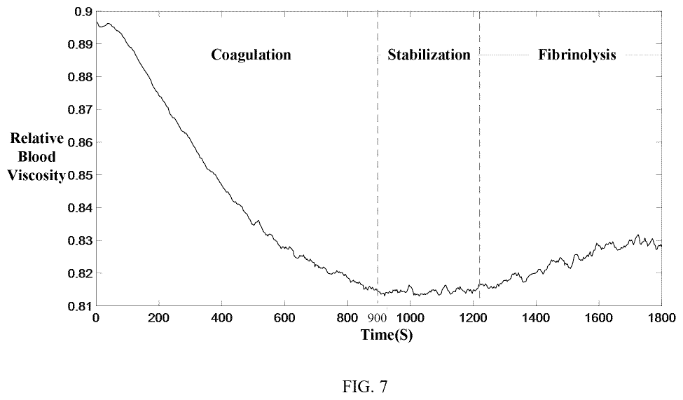

Results in FIG. 7 generally align with a blood coagulation-fibrinolysis process: the first 15 minutes represent blood coagulation, the 15^{th} to 20^{th} minutes show a stable phase, and the 20^{th} to 30^{th} minutes indicate fibrinolysis. The device of the present disclosure achieves rapid blood viscoelasticity measurement while preventing blood sample contact with air and avoiding probe rotation and immersion into the blood sample. Compared with conventional techniques, the method of the present disclosure provides higher accuracy.

### Comparative Example 1

The method of the present disclosure and the measurement method provided by in Chinese Patent Application CN113203661A are respectively used to determine true values of a standard viscosity fluid for comparison.
1) A functional relationship between a viscosity value and a relative attenuation rate obtained through simulation is:

$$y = a * {x}^{b} + c$$

2) For measured viscosity values of 4.5 cp, 98.5 cp, and 476 cp, corresponding relative attenuation rates are 0.997, 0.906, and 0.857, respectively. Substituting these values into the function yields:

$$y = - 0.03531 * {x}^{0.2766} + 1.052$$

3) For measured viscosity values of 9.2 cp and 346 cp, corresponding relative attenuation rates are 0.987 and 0.876, respectively. Substituting the attenuation rates into the above equation yields viscosity values of 9.1 cp and 334.1 cp, with errors of 1.38% and 3.43%, respectively.
4) According to the measurement method provided by the Chinese Patent Application CN113203661A, measured viscosity values of 9.2 cp and 346 cp yield calculated values of 10.1 cp and 372 cp, with errors of 9.7% and 7.5% respectively.

The experimental comparisons demonstrate higher accuracy of the method provided by the present disclosure.

The present disclosure employs a torsional guided wave for measurement, which exhibits non-dispersive characteristics within specific frequency ranges and high sensitivity to fluid viscosity changes. The method resists interference from other factors during measurement, achieving micro-volume, rapid, and non-immersive blood viscoelasticity measurement.

The above specific implementations are intended to explain the present disclosure, rather than to limit the present disclosure. Within the spirit of the present disclosure and the protection scope of the claims, any modification and change to the present disclosure should fall into the protection scope of the present disclosure.

What is claimed is:

1. A torsional guided wave-based blood viscoelasticity measurement device using a capillary metal tube, comprising:
a housing, a capillary metal tube, a signal receiving module, and a signal excitation module, wherein
the capillary metal tube is disposed inside the housing, and is configured to hold a blood sample;
the signal excitation module is disposed at a first end of the capillary metal tube, and comprises a cuboid permanent magnet and an electrode; the cuboid permanent magnet is configured to provide a static bias magnetic field; and the electrode is configured to conduct a current and apply a dynamic induced magnetic field to the capillary metal tube; and
the signal receiving module is disposed at a second end of the capillary metal tube, and comprises a cylindrical permanent magnet and a receiving coil; the cylindrical permanent magnet is disposed at an end portion of the capillary metal tube; and the receiving coil is disposed adjacent to the end portion of the capillary metal tube.

2. The torsional guided wave-based blood viscoelasticity measurement device using the capillary metal tube according to claim 1, further comprising a microcontroller, a guided wave excitation device, a pulse generation device, a power amplification device, an echo receiving module, a preamplifier module, a data acquisition module, and a display module, wherein
the receiving coil is connected to the microcontroller through the echo receiving module, the preamplifier module, and the data acquisition module in sequence; the microcontroller is connected to the electrode through the guided wave excitation device, the pulse generation device, and the power amplification device in sequence; and the microcontroller is connected to the display module.

3. The torsional guided wave-based blood viscoelasticity measurement device using the capillary metal tube according to claim 1, wherein
the housing is further internally provided with a heating layer, an insulation layer, a temperature probe, and a temperature controller;
the heating layer is disposed outside the capillary metal tube, is connected to the capillary metal tube through contact, and is electrically connected to the microcontroller;
the insulation layer contacts and wraps around the heating layer;
the temperature probe is disposed at the first end of the capillary metal tube where the electrode is located; and
the temperature controller is electrically connected to the microcontroller through the temperature probe.

4. The torsional guided wave-based blood viscoelasticity measurement device using the capillary metal tube according to claim 1, wherein the signal receiving module is disposed at the second end of the capillary metal tube; the cylindrical permanent magnet is directly connected to an end face of the capillary metal tube through magnetism; the receiving coil is sleeved outside the capillary metal tube, wherein an inner wall of the receiving coil does not contact an outer wall of the capillary metal tube, and a difference between an inner diameter of the receiving coil and an outer diameter of the capillary metal tube is less than 1.0 mm; and the capillary metal tube is directly detachable and replaceable through a plug-and-pull method.

5. The torsional guided wave-based blood viscoelasticity measurement device using the capillary metal tube according to claim 1, wherein the capillary metal tube is made of a magnetic material; and the cylindrical permanent magnet is magnetically adsorbed onto the end portion of the capillary metal tube.

6. The torsional guided wave-based blood viscoelasticity measurement device using the capillary metal tube according to claim 1, wherein
the capillary metal tube is made of a material, comprising pure nickel, carbon steel, iron-cobalt alloy, iron-aluminum alloy, and iron-cobalt-nickel alloy.

7. The torsional guided wave-based blood viscoelasticity measurement device using the capillary metal tube according to claim 1, wherein
the end portion of the capillary metal tube is provided with a rubber plug for sealing.

8. A torsional guided wave-based blood viscoelasticity measurement method using a capillary metal tube, applied to the torsional guided wave-based blood viscoelasticity measurement device according to claim 1, and comprising the following steps:
1) calculating a guided wave dispersion curve of the capillary metal tube based on a structural geometric parameter and a material mechanical characteristic of the capillary metal tube; and selecting an excitation frequency based on the guided wave dispersion curve;
2) exciting the electrode on the capillary metal tube when the capillary metal tube is empty, and generating a torsional guided wave that propagates reciprocally between two end faces of the capillary metal tube until energy is depleted, wherein the capillary metal tube has a length of l m; collecting, by the receiving coil, a total of 2 klm guided wave signals from first k reciprocations as an empty-tube reference signal w₀;
3) filling a blood sample into the capillary metal tube; acquiring an echo signal in the same manner as the step 2) as a blood-filled test signal, wherein an i-th blood-filled test signal is denoted as wᵢ,
4) extracting envelopes from the empty-tube reference signal w₀ and each blood-filled test signal wᵢ respectively to obtain envelope signals; taking n echo peaks from each of the envelope signals for fitting, and obtaining attenuation rates b₀ and b₁ of the n echo peaks, respectively; and determining, based on the attenuation rates b₀ and b₁ and a pre-calibrated relationship between a relative blood viscosity of the blood sample and the attenuation rate, a relative blood viscosity corresponding to each blood-filled test signal wᵢ; and
5) setting a sampling count m and a sampling interval Δt, filling the blood sample into the capillary metal tube, and continuously repeating the steps 3) and 4) to perform sampling and obtain a relative blood viscosity for each test; and plotting a time-dependent relative blood viscosity curve of the blood sample over a duration of m*Δt as a thrombelastogram, wherein a blood viscoelasticity measurement is achieved.

9. The torsional guided wave-based blood viscoelasticity measurement method using the capillary metal tube according to claim 8, wherein in the step 4), the step of taking the n echo peaks from each of the envelope signals comprises: selecting, based on the envelope signal, a first threshold l₁ and a second threshold l₂, with an interval being l=l₁−l₂; establishing, starting from an initial point x₀ of the envelope signal, n consecutive segments, each with a length of l, wherein the n consecutive segments are [x₀,x₀+l], [x₀+l,x₀+2l], . . . , [x₀+ (n−1)l,x₀+nl], and each of the n consecutive segments comprises only one peak; and extracting the peak from each of the n consecutive segments as an echo peak, and extracting all peaks from the n consecutive segments as finally selected n echo peaks.

10. The torsional guided wave-based blood viscoelasticity measurement method using the capillary metal tube according to claim 8, wherein in the step 4), the attenuation rate is derived by fitting as follows:
$$y = a * {b}^{x}$$
wherein x denotes a sequence number of the echo peak; y denotes a value of the echo peak; b denotes the attenuation rate of the echo peak; and a denotes an amplitude normalization coefficient.

11. The torsional guided wave-based blood viscoelasticity measurement device using the capillary metal tube according to claim 5, wherein
the capillary metal tube is made of a material, comprising pure nickel, carbon steel, iron-cobalt alloy, iron-aluminum alloy, and iron-cobalt-nickel alloy.

12. The torsional guided wave-based blood viscoelasticity measurement method according to claim 8, wherein the torsional guided wave-based blood viscoelasticity measurement device further comprises a microcontroller, a guided wave excitation device, a pulse generation device, a power amplification device, an echo receiving module, a preamplifier module, a data acquisition module, and a display module, wherein
the receiving coil is connected to the microcontroller through the echo receiving module, the preamplifier module, and the data acquisition module in sequence; the microcontroller is connected to the electrode through the guided wave excitation device, the pulse generation device, and the power amplification device in sequence; and the microcontroller is connected to the display module.

13. The torsional guided wave-based blood viscoelasticity measurement method according to claim 8, wherein in the torsional guided wave-based blood viscoelasticity measurement device, the housing is further internally provided with a heating layer, an insulation layer, a temperature probe, and a temperature controller;
the heating layer is disposed outside the capillary metal tube, is connected to the capillary metal tube through contact, and is electrically connected to the microcontroller;
the insulation layer contacts and wraps around the heating layer;
the temperature probe is disposed at the first end of the capillary metal tube where the electrode is located; and
the temperature controller is electrically connected to the microcontroller through the temperature probe.

14. The torsional guided wave-based blood viscoelasticity measurement method according to claim 8, wherein in the torsional guided wave-based blood viscoelasticity measurement device, the signal receiving module is disposed at the second end of the capillary metal tube; the cylindrical permanent magnet is directly connected to an end face of the capillary metal tube through magnetism; the receiving coil is sleeved outside the capillary metal tube, wherein an inner wall of the receiving coil does not contact an outer wall of the capillary metal tube, and a difference between an inner diameter of the receiving coil and an outer diameter of the capillary metal tube is less than 1.0 mm; and the capillary metal tube is directly detachable and replaceable through a plug-and-pull method.

15. The torsional guided wave-based blood viscoelasticity measurement method according to claim 8, wherein in the torsional guided wave-based blood viscoelasticity measurement device, the capillary metal tube is made of a magnetic material; and the cylindrical permanent magnet is magnetically adsorbed onto the end portion of the capillary metal tube.

16. The torsional guided wave-based blood viscoelasticity measurement method according to claim 8, wherein in the torsional guided wave-based blood viscoelasticity measurement device, the capillary metal tube is made of a material, comprising pure nickel, carbon steel, iron-cobalt alloy, iron-aluminum alloy, and iron-cobalt-nickel alloy.

17. The torsional guided wave-based blood viscoelasticity measurement method according to claim 8, wherein in the torsional guided wave-based blood viscoelasticity measurement device, the end portion of the capillary metal tube is provided with a rubber plug for sealing.

18. The torsional guided wave-based blood viscoelasticity measurement method according to claim 15, wherein in the torsional guided wave-based blood viscoelasticity measurement device, the capillary metal tube is made of a material, comprising pure nickel, carbon steel, iron-cobalt alloy, iron-aluminum alloy, and iron-cobalt-nickel alloy.

19. The torsional guided wave-based blood viscoelasticity measurement method using the capillary metal tube according to claim 12, wherein in the step 4), the step of taking the n echo peaks from each of the envelope signals comprises: selecting, based on the envelope signal, a first threshold l₁ and a second threshold l₂, with an interval being l=l₁−12; establishing, starting from an initial point x₀ of the envelope signal, n consecutive segments, each with a length of l, wherein the n consecutive segments are [x₀,x₀+l], [x₀+l,x₀+2l], . . . , [x₀+ (n−1)l,x₀+nl], and each of the n consecutive segments comprises only one peak; and extracting the peak from each of the n consecutive segments as an echo peak, and extracting all peaks from the n consecutive segments as finally selected n echo peaks.

20. The torsional guided wave-based blood viscoelasticity measurement method using the capillary metal tube according to claim 13, wherein in the step 4), the step of taking the n echo peaks from each of the envelope signals comprises: selecting, based on the envelope signal, a first threshold l₁ and a second threshold l₂, with an interval being l=l₁−l₂; establishing, starting from an initial point x₀ of the envelope signal, n consecutive segments, each with a length of l, wherein the n consecutive segments are [x₀,x₀+l], [x₀+l,x₀+2l], . . . , [x₀+(n−1)l,x₀+nl], and each of the n consecutive segments comprises only one peak; and extracting the peak from each of the n consecutive segments as an echo peak, and extracting all peaks from the n consecutive segments as finally selected n echo peaks.
